(19) United States
(12) Patent Application Publication
(10) Pub. No.: US 2009/0081312 A1
(43) Pub. Date: Mar. 26, 2009

(54) PREVENTION AND TREATMENT OF ALVEOLAR OSTEITIS

(76) Inventors: James C. Block, Minnetonka, MN (US); Gary R. Jernberg, North Mankato, MN (US)

Correspondence Address: MERCHANT & GOULD PC, P.O. BOX 2903, MINNEAPOLIS, MN, 55402-0903, US

(21) Appl. No.: 12/055,518

(22) Filed: Mar. 26, 2008

Related U.S. Application Data
(63) Continuation-in-part of application No. 11/690,992, filed on Mar. 26, 2007.

(51) Int. Cl. A61K 33/38; A61P 19/00

(52) U.S. Cl. 424/618

(57) ABSTRACT

A tissue adhesive antimicrobial material that is placed into a tooth extraction site for the sustained release of silver for the prevention and treatment of alveolar osteitis. The antimicrobial material is placed into a tooth extraction site via syringe, hand instrument or hand delivery device.

### CROSS REFERENCE TO RELATED APPLICATIONS

This application is a continuation-in-part of U.S. patent application Ser. No. 11/690,992 filed Mar. 26, 2007, which application is hereby incorporated by reference in its entirety.

### TECHNICAL FIELD

The present invention relates to compositions and methods of preventing and treating alveolar osteitis utilizing a sustained, controlled release of antimicrobial silver agent at the site of tooth extraction.

### BACKGROUND

Alveolar osteitis (the colloquial term is dry socket) is a complication of tooth extraction leading to a delayed healing of an extraction site. It is a very painful condition. The patient generally describes the situation as an aching or throbbing in their jaw. Clinical examination shows an increase of edema (swelling) and erythema (redness) in the area of the extraction and exposed bone in the socket of the extracted tooth. Many times a bad odor is associated with alveolar osteitis and patients complain of a bad taste or oozing from the socket.

The incidence of alveolar osteitis has been reported as 3 to 4 percent for routine extractions. See A. J. McGregor, Treatment of dry socket by general practitioners, Brit. Dent. J. 156:132 (1984). It has been reported up to 31 percent for the surgical removal of impacted mandibular third molars. See J. Rud, Removal of impacted lower third molars with acute pericoronitis and necrotizing gingivitis, Brit. J. Oral Surgery 7:153 (1970). Alveolar osteitis results in burdensome costs to the healthcare system in terms of the time and effort for treatment. It also results in loss of work or school time for the patient.

Alveolar osteitis is characterized by premature breakdown of the fibrin clot. The fibrin clot functions as a transitory scaffold for cell adhesion and migration during early wound healing. For many years it was thought that alveolar osteitis was caused by an inadequate or depressed coagulation system or mechanical factors that caused loss of the blood clot in the extraction socket. Infection at the time of the extraction, the difficulty of tooth extraction, the amount of time required for the extraction and various other factors including smoking, placing foreign objects in the socket, scraping the socket or eating a chewy diet have all been implicated as causative factors.

A classic series of articles provided a basis for the pathophysiology of the fibrin clot loss associated with alveolar osteitis. See H. Birn, Bacteria and Fibrinolytic activity in “dry socket”, Acta. Odont. Scand. 28:773 (1970); Fibrinolytic activity of alveolar bone in “dry socket”, Acta. Odont. Scand. 30:32 (1973) and Etiology and pathogenesis of fibrinolytic alveolitis (“dry socket”), Int. J. Oral Surg. 2:211 (1973). Subsequent studies have suggested that both bacteria and local fibrinolysis are involved in alveolar osteitis pathogenesis. Recently, the first comprehensive assessment of bacteria associated alveolar osteitis versus normally healing extraction wounds was made. See H. J. Shiau, Microbiota Associated with normal healing extraction wounds and alveolar osteitis, IADR 83^{rd} General Session (2005). Results showed Gemella haemolysans, Gemella sanguinis, Cardiobacterium hominis and Campylobacter graci/is at a significantly greater frequency in alveolar osteitis sites than normal healing extraction sites. Streptococcus intermedius and Prevotella intermedia demonstrated an ability to lyse fibrin in vitro.

Over the years many different materials and methods have been used for treating alveolar osteitis. None of these, including the use of systemic antibiotics, have been particularly effective.

It is our contention that alveolar osteitis is a localized infection with a multi-factorial bacterial etiology. Further, we contend that alveolar osteitis is a bacterial biofilm infection with inherent resistance to conventional treatment methods. (e.g., systemic antibiotics).

Accordingly, there is a need for improved delivery of antibacterial agents to the tooth extraction site for the prevention and treatment of alveolar osteitis.

### SUMMARY

The present invention relates to the use of antimicrobial silver agents for sustained, controlled and targeted release via placement into the alveolar socket (tooth extraction socket) for the prevention and treatment of alveolar osteitis.

In one embodiment of the present invention, a therapeutic composition of time-release silver within a polymeric carrier is provided. In another embodiment of the present invention, silver is contained within nanoparticles or microparticles. In yet another embodiment of the present invention, silver is in the form of a silver salt such as silver chloride.

In one embodiment, a matrix configuration of the polymeric carrier provides for a sustained, programmed release of silver to the intended tissue surface. In still another embodiment of the present invention, the active silver cation is released to the intended tissue surface as the associated salt anion is taken up by the intended tissue. In yet another embodiment of the present invention, the therapeutic composition is placed into the alveolar socket via syringe or hand delivery instrument or device.

In an embodiment of the present invention, the therapeutic composition is in the form of a gel, paste, viscid liquid, semi-solid or some combination thereof. In another embodiment of the present invention, the polymeric carrier is biocompatible. In another embodiment, the polymeric carrier is biodegradable. In yet another embodiment, the polymeric carrier is bioadhesive.

These and various other advantages and features of novelty which characterize the invention are pointed out with particularity in the claims annexed hereto and forming a part hereof. However, for a better understanding of the invention, its advantages, and objects attained by its use, reference should be made to the drawings which form a further part hereof, and to the accompanying descriptive matter, in which there is illustrated and described preferred embodiments of the invention.

### BRIEF DESCRIPTION OF THE DRAWINGS

FIG. 1 is a diagrammatic view of a tooth in the jaw.

FIG. 2 is a diagrammatic view of a tooth extraction site.

FIG. 3 is a diagrammatic view of the antimicrobial material being placed into the tooth extraction site via syringe.

### DETAILED DESCRIPTION

Various embodiments of the present invention will be described in detail with reference to the drawings, wherein like reference numerals represent like parts throughout the several views. Reference to various embodiments does not limit the scope of the invention, which is limited only by the scope of the claims attached hereto. Additionally, any examples set forth in this specification are not intended to be limiting and merely set forth some of the many possible embodiments for the claimed invention.

All publications and patent applications in this specification are indicative of the level of ordinary skill in the art to which this invention pertains and are incorporated herein by reference in their entireties.

In the drawings in which like reference numerals and letters indicate corresponding parts throughout the several views: Referring now to the figures, FIG. 1 is a diagrammatic illustration of a tooth 10 embedded in alveolar bone 11 with ligaments 12 attaching the root 13 to the alveolar bone 11. The crown 14 emerges from the alveolar bone through the gingiva (gum tissue) 15 into the oral cavity.

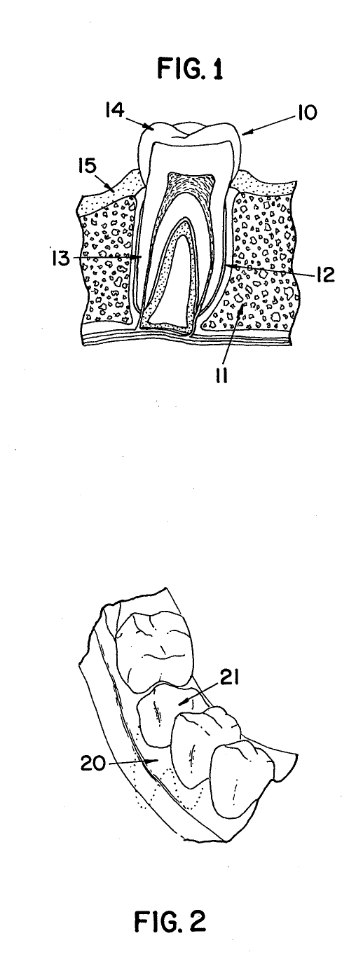

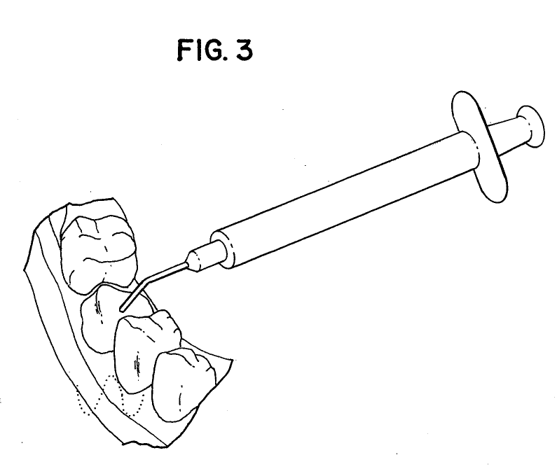

FIG. 2 illustrates a tooth extraction site where the alveolar bone 20 is exposed to the oral cavity. Numerous oral bacteria can gain access to the tooth extraction site and potentially cause an infection. If the blood clot is lost the bacteria form a biofilm infection that layers the alveolar socket 21.

FIG. 3 illustrates diagrammatically a tooth extraction site where a therapeutic composition is deposited into an alveolar socket via syringe following tooth extraction. The therapeutic composition can be delivered either as a preventive directly following tooth extraction or as a treatment for alveolar osteitis after it occurs.

Accordingly, the present invention provides compositions and methods for the prevention or treatment of alveolar osteitis. Specifically, in a first aspect, the present invention provides a method of preventing or treating alveolar osteitis including combining antimicrobial silver with a polymeric carrier agent to form a therapeutic composition. The therapeutic composition is applied to the tooth extraction socket wherein the antimicrobial silver is sustainably released to the alveolar bone in the tooth extraction socket.

In embodiments, the present invention provides a method of preventing or treating alveolar osteitis with a composition containing time-release silver within a polymeric carrier. Silver (particularly ionic silver) is known to those of skill in the art as an effective antimicrobial agent, because it has broad-spectrum antimicrobial action, it is well tolerated by tissues and is generally compatible with materials commonly used in therapeutic compositions.

Although not limiting to the present invention, it is believed that in solution, silver is present in its cationic form and has high affinity for the negatively charged side chains on biological molecules, such as sulfhydryl, carboxyl, phosphate and other charged groups. Once the silver cation is bound to these groups, the structure of the biomolecule is altered and it no longer has pathogenic function. Silver can simultaneously attack multiple sites within a cell to inactivate critical physiological functions such as cell wall synthesis, membrane transport, RNA and DNA synthesis and translation, protein folding, protein function, etc. Because silver can impact multiple cell functions at the same time, it is not possible for the pathogenic organism to mutate to avoid the antimicrobial effects of the silver. Furthermore, because silver can affect many different functions of the pathogenic organism, it has nonselective broad-spectrum antimicrobial activity against many microorganisms including bacteria, fungi and yeast. Against many microorganisms, silver is more efficient than traditional antibiotics because the silver ion is active even at very low concentration or quantity. For example, as little as one part per billion (ppb) may be sufficient to prevent cell bacterial cell growth (see B Gibbins, Ostomy Wound Management 49: 5-6 (2003)).

Silver has increased antimicrobial activity when either metallic silver, or a silver salt, is present in an aqueous or liquid form, wherein the metallic silver is readily oxidized into the active monovalent cationic form (Ag⁺). The ionic form is limited in that it is only active for a short duration of time (usually a few days). Antimicrobial silver therefore can be advantageously provided in a form with persistent activity. Accordingly, in another aspect of the invention, antimicrobial silver is provided as, or contained within, nanoparticles or microparticles. Metallic silver is incorporated onto the surface of a microparticle or nanoparticle by methods known to those of skill in the art. The metallic silver is then activated by exposure to moisture, when the nanoparticle or microparticle comes in contact with body fluids. The silver on the surface of the particle is slowly oxidized into the active ionic form, and because of this slow oxidation, the antimicrobial effect of the silver lasts significantly longer than with ionic silver in solution in contact with a wound site. The incorporation of metallic silver onto or into microparticles or nanoparticles thus provides a controlled and sustained release of the active silver cation.

In embodiments, the present invention provides a method of preventing or treating alveolar osteitis with a composition containing time-release silver within a polymeric carrier. In an aspect, the silver is in the form of a silver salt such as silver chloride (AgCl), silver bromide (AgBr), silver carbonate (Ag₂CO₃), silver phosphate (Ag₃PO₄, silver oxide (Ag₂O), or other salts of silver. The silver salt is provided as, or contained within, nanoparticles or microparticles. A monolayer of a silver salt (such as silver oxide or silver chloride) can be formed on the surface of a nanoparticle or microparticle. When the nanoparticle or microparticle comes in contact with an aqueous environment, the salt monolayer slowly dissolves and Ag⁺ ions are released in solution. Because of the slow dissolution of the salt monolayer, the antimicrobial silver is released in a controlled and sustained manner, and the effect lasts significantly longer than simply introducing a solution of silver salt into a wound site.

Although not limiting to the present invention, it is believed that the antimicrobial efficacy of silver in the treatment of alveolar osteitis depends on the method of delivery, or the system used for delivery. Therefore, in an embodiment, the antimicrobial silver is provided in a matrix configuration of the polymeric carrier, for a sustained, programmed release of silver to the alveolar socket surface. The polymeric carrier can facilitate the delivery of the silver agent into the alveolar socket. It also facilitates retention of the silver agent in the alveolar socket. Further, it allows for sustained, time-release of active silver within the alveolar socket. Further still, it facilitates deposition of the active silver to the alveolar surface. Preferred carrier agents include polymeric carrier agents that are biocompatible, preferably biodegradable, preferably tissue bioadhesive and preferably bone adhesive.

In an aspect, the polymer carrier can be in the shape of a powder, film, fiber, granules used to incorporate the antimicrobial silver. In a preferred aspect, the antimicrobial silver is provided in a nanoparticulate or microparticulate polymer carrier matrix. The incorporation of silver onto or into microparticles or nanoparticles of a biodegradable and bioadhesive polymer carrier provides a controlled and sustained release of the active silver cation.

In an aspect, the polymer carrier for the antimicrobial silver can include synthetic or natural polymers, or combinations of synthetic and natural polymers. Such polymers include, without limitation, aliphatic and aromatic polyesters, liquid crystalline polymers for high performance resins and fibers, polyester block copolymers, aliphatic and aromatic polyamides, including various nylons, copolymerized polyamides, polyolefins and polyolefin copolymers, vinyl polymers, fluorocarbon polymers, polyurethanes, polyketones, polyethers, polysulfides, polysulfones, polysiloxanes, polycarbonates, synthetic thermosetting resins, natural polymers such as cellulosic materials, cotton and wool, semi-synthetic polymers such as rayon, acetate rayon, etc., and polysaccharides. In a preferred aspect, the polymer carrier for the antimicrobial silver includes polylactides, polyglycolides, polylactide-co-glycolides, polycaprolactones, polyalkyl cyanoacrylates, polyorthoesters, polyphosphoesters, polyanhydrides and polyphosphazenes.

In an aspect, the polymeric carrier is a biocompatible polymer. A biocompatible substance is a substance capable of implantation or use with biological systems without a major immunological response, i.e. a substance where implantation in the body does not cause excessive fibrosis or rejection reactions. In a preferred aspect, the biocompatible polymer carrier for the antimicrobial silver includes polylactides, polyglycolides, polylactide-co-glycolides, polycaprolactones, polyalkyl cyanoacrylates, polyorthoesters, polyphosphoesters, polyanhydrides and polyphosphazenes.

In another aspect, the polymer carrier for the antimicrobial silver is a biodegradable polymer. The rationale for using biodegradable (or bioerodible) polymers is that these polymers are absorbed by the body and need not be removed surgically. Biodegradation occurs over time, as the polymeric material is converted from a water-insoluble substance to a water-soluble substance. In an aspect, a variety of natural, synthetic, and biosynthetic polymers that are biodegradable are used as carriers. Biodegradable polymers include heteroatom-containing polymer, typically with chemical linkages such as anhydride, ester, or amide bonds, for example. Biodegradable polymers useful as carriers for antimicrobial silver include poly(esters) based on polylactide (PLA), polyglycolide (PGA), polycaprolactone (PCL). When these materials biodegrade, the corresponding hydroxy acid is formed, making the polymers safe for in vivo use. Other biodegradable polymers include poly(hydroxyalkanoate)s of the PHB-PHV class, additional poly(ester)s, and natural polymers, particularly, modified poly(saccharide)s, such as starch, cellulose, and chitosan, for example.

In yet another aspect, the polymer carrier is bioadhesive. A bioadhesive polymer is a polymeric material capable of adhering to mucosal surfaces. In an aspect, the bioadhesive polymer acts as a barrier that prevents immediate release of the antimicrobial silver immediately on application. Over time, the bioadhesive polymer carrier slowly degrades or erodes, releasing the antimicrobial silver in a sustained, controlled manner. Bioadhesive polymer carriers used with antimicrobial silver include cellulose-based polymers, such as hydroxyethyl cellulose and hydroxypropyl cellulose, for example; ethylene glycol polymer and its copolymers, oxyetheylene polymers, polyvinyl alcohol, polyvinyl acetate, and esters of hyaluronic acid. In a preferred aspect, the bioadhesive polymers include hydroxyethyl cellulose, hydroxypropyl cellulose, hydroxypropylmethyl cellulose, hydroxyethylmethyl cellulose, polyvinylalcohol, polyethylene glycol, polyethylene oxide, ethylene oxide-propylene oxide co-polymers, or combinations thereof.

In an embodiment, the active silver cation is released to the alveolar socket surface as the associated salt anion is taken up by the alveolar socket surface tissue. Antimicrobial silver (in salt form) is incorporated into a microparticulate or nanoparticulate carrier. When the nanoparticle or microparticle comes in contact with an aqueous environment, the salt slowly dissolves and Ag⁺ ions (silver cations) are released into solution in the alveolar socket surface.

In an aspect, the therapeutic composition is placed into the alveolar socket via syringe or hand delivery instruments or device. In another aspect, the therapeutic composition is in the form of a gel, paste, viscid liquid, semi-solid or some combination thereof.

It will be appreciated that other substances such as diluents, solvents, fillers, flavorings, stabilizers or other ingredients utilized to facilitate the combination, handling or other properties of the therapeutic treatment compositions may be required.

It will be appreciated that the particular therapeutic compositions of the present invention, as well as the dosages and durations of treatment will be in accordance with accepted treatment.

### EXAMPLES

### Example 1-Efficacy of Antimicrobial Wound Dressings Against Oral Bacteria

Dressings impregnated with antimicrobial compounds are commercially available, but the efficacy against oral bacteria has not previously been tested. In the following example, various antimicrobial compounds are tested using an in vitro model that simulates a tooth extraction socket exposed to oral bacteria. The unexpected results shown in the following example demonstrate that only certain silver-containing materials appear to be completely effective against particular microorganisms, and illustrates an advantageous method of delivering silver.

### Materials and Methods

Sterile 5 ml polystyrene test tubes were utilized in the in vitro assay. One ml of sterile bacterial growth medium (Brain Heart Infusion broth, BHI) was added to each tube. Sterile wound dressings were then shaped into a bolus large enough to occlude the test tube, and pushed down until they touched the surface of the growth medium. Various dressings were compared, including (1) cotton gauze (with petroleum jelly, but without any antimicrobial material; negative control); (2) iodoform gauze (with petroleum jelly, but without any additional antimicrobial material); (3) iodoform gauze impregnated with a medicated gel containing eugenol (the current standard of care); (4) cotton gauze impregnated with an antimicrobial gel (Silvasorb gel); (5) iodoform gauze impregnated with the same antimicrobial gel (Silvasorb gel), and (6) nylon fabric with an antimicrobial coating (Acticoat or Silverrol).

One ml of bacterial suspension was added on top of each dressing. The suspension consisted of Gemella haemolysans, an oral species that has been recovered from tooth extraction sites. The species is grown in BHI to an optical density of 1.0. As a control for contamination, one 5 ml test tube containing plain gauze received sterile BHI. The test tubes were incubated in an anaerobic chamber at 37° C. for 48-72 hours. The media in the bottom of the tubes was then sampled, taking care to avoid contamination. Ten-fold serial dilutions were then plated on blood agar. The plates were incubated as before, and colony-forming units (CFU) were determined for each test tube. Replicate experiments were run in triplicate. To determine which dressing was the most effective in preventing the passage of G. haemolysans into the bottom of the tube, mean CFUs for each test dressing were determined (from the triplicate trials) and compared by analysis of variance followed by the student Newman-Keuls test. The results for each dressing is shown in Table I.

TABLE I

| | Mean Bacterial |
|---|---|
| Treatment | Growth (CFU) |
| Gauze without G. haemolysans | 0 |
| Gauze with G. haemolysans | 1.30E+10 |
| Gauze with petroleum jelly | 2.22E+08 |
| Iodoform with petroleum jelly | 0 |
| Iodoform packing strip with eugenol | 0 |
| Gauze with Silvasorb gel | 9.74E+08 |
| Iodoform strip with Silvasorb gel | 0 |
| Acticoat silver-coated dressing | 0 |
| Silverrol would packing strip (tube form) | 8.88E+08 |

### Results

No growth was seen in the contamination control tube, i.e. gauze with sterile medium, indicating that contamination was not a problem with this in vitro system. In contrast, very extensive growth occurred on gauze exposed to G. haemolysans, with CFUs of about 10 logs. This positive control confirmed that bacterial passage and growth were not impeded by the gauze. Iodoform gauze without any additional antimicrobial agent showed a substantial antimicrobial effect, reducing GFUs by about 4 logs, and a synergistic effect was observed when the iodoform strip was impregnated with the standard-of-care dressing containing eugenol in a base of white petrolatum, with complete elimination of bacterial growth. This effect was also seen with the iodoform gauze impregnated with petroleum jelly, indicating that the petrolatum itself could be acting as a physical barrier to bacterial passage. However, substantial growth (about eight logs) was seen with plain cotton gauze impregnated with petroleum jelly, suggesting that the iodine in the iodoform gauze (and not the petroleum jelly) was responsible for complete inhibition of bacterial growth. Similarly, while the plain gauze impregnated with antimicrobial silver (Silvasorb gel) showed substantial bacterial growth, the iodoform strip impregnated with Silvasorb showed complete inhibition. Thus, only minimal inhibition is attributable to the Silvasorb, and the inhibition of bacterial growth is primarily due to iodine.

Of the two silver fabric dressings, Silverrol showed significant growth of bacterial (about eight logs). In contrast, the Acticoat dressings showed complete inhibition of growth, and was the only silver-containing material that was completely effective in blocking passage and growth of G. haemolysans. This suggests that the antimicrobial efficacy of the silver may depend on the method of delivery.

It is to be understood, however, that even though numerous characteristics and advantages of the invention have been set forth in the forgoing description, together with details of the structure and function of the invention, the disclosure is illustrative only and changes may be made in detail, especially in matters of shape, size and arrangement of parts within the principle of the invention, to the full extent indicated by the broad general meaning of the terms in which the appended claims are expressed.

The various embodiments described above are provided by way of illustration only and should not be construed to limit the invention. Those skilled in the art will readily recognize various modifications and changes that may be made to the present invention without following the example embodiments and applications illustrated and described herein, and without departing from the true spirit and scope of the present invention without following the example embodiments and applications illustrated and described herein, and without departing from the true spirit and scope of the present invention, which is set forth in the following claims.

1. A method of treating alveolar osteitis comprising administering a composition comprising antimicrobial silver and a polymeric carrier agent.

2. A method according to claim 1, wherein the antimicrobial silver is in the form of nanoparticles or contained within microspheres.

3. A method according to claim 1, wherein the antimicrobial silver is in the form of one or more silver salts.

4. A method according to claim 3, wherein the antimicrobial silver salt is silver chloride, silver bromide, silver carbonate, silver phosphate, silver oxide, or mixture thereof.

5. A method according to claim 3, wherein the antimicrobial silver cations are released to the alveolar socket surface as the associated salt anions are taken up by the alveolar socket surface tissue.

6. A method according to claim 1, wherein the polymeric carrier agent provides sustained release of antimicrobial silver to the alveolar socket surface.

7. A method according to claim 6, wherein the polymeric carrier agent forms a biocompatible matrix for the antimicrobial silver agent.

8. A method according to claim 6, wherein the polymeric carrier is biodegradable.

9. A method according to claim 8 wherein the biodegradable polymeric carrier comprises polylactide, polyglycolide, polylactide-co-glycolide, polycaprolactone, polyalkyl cyanoacrylate, polyorthoester, polyphosphoester, polyanhydrides, or polyphosphazenes, copolymer thereof, or mixture thereof.

10. A method according to claim 6, wherein the polymeric carrier is bioadhesive.

11. A method according to claim 10, wherein the polymeric carrier comprises cellulose-based polymer, ethylene glycol polymer, a copolymer of ethylene glycol, oxyetheylene polymer, polyvinyl alcohol, polyvinyl acetate, ester of hyaluronic acid, copolymer thereof, or mixture thereof.

12. A method according to claim 1, wherein the therapeutic treatment composition is in the form of a semi-solid, a paste, a liquid, a gel or combination thereof.

13. A method according to claim 1, wherein the therapeutic treatment composition is placed into the alveolar socket via syringe, hand instrument or delivery device.
